(19) United States
(12) Patent Application Publication
(10) Pub. No.: US 2007/0112600 A1
(43) Pub. Date: May 17, 2007

(54) System for massaging a person

(76) Inventors: David A. Palmer, San Francisco, CA (US); Samuel A. Keller, San Francisco, CA (US); Jonathan S. Bruck, San Francisco, CA (US)

Correspondence Address: Stephen M. De Klerk;BLAKELY, SOKOLOFF, TAYLOR & ZAFMAN LLP, Seventh Floor, 12400 Wilshire Boulevard, Los Angeles, CA, 90025-1026, US

(21) Appl. No.: 11/595,069

(22) Filed: Nov. 10, 2006

Related U.S. Application Data
(60) Provisional application No. 60/735,400, filed on Nov. 11, 2005.

(51) Int. Cl. G06Q 10/00

(52) U.S. Cl. 705/2; 705/5

(57) ABSTRACT

The invention provides a system for massaging a person. A removable structure provides an area to receive a massage. A supporting apparatus supports the person receiving a massage. An electronic scheduling system enables the person to schedule the massage.

This application is a non-provisional application of provisional application U.S. 60/735,400 filed on Nov. 11, 2005.

### BACKGROUND

1.) Field of the Invention

The invention relates to systems for massaging a person.

2.) Discussion of Related Art

People often suffer from mental and physical stress, and anxiety. Physical massage is an excellent approach to reducing stress and anxiety.

Scheduling massages and finding suitable massage vendors is also problematic. Traditionally massages are typically available in massage parlors or exclusive resorts. Massage parlors have a negative connotation associated with them, and thus societal stigma may be attached to massages given in exclusively private areas. Resorts are not stigmatized but are typically only available to very wealthy consumers or consumers on vacation destinations, which places the availability of stress reducing massage out of reach to most consumers. Traditional massage also usually requires the consumer to be at least partially undressed and lying fully horizontal on a massage table.

Professional chair massage has been available in public thoroughfares such as malls, airports, conventions centers, in limited varieties. Public massages are typically given in massage chairs with the customer fully dressed. However massages available at public thoroughfares are not typically private and given in plain view. Consumers desire at least some privacy when receiving a massage. Availability and scheduling of professional chair massage is also problematic because chair massage usually only services walk-in customers.

### SUMMARY OF THE INVENTION

The invention provides a system for massaging a person including an intactly removable structure with at least a first wall structure defining at least a partial enclosure; and a supporting apparatus within the enclosure for supporting the person in a relatively upright and face downward position for applying pressure to select locations on the person, wherein the supporting apparatus directly supports a face of the person.

The first wall structure may form a semi enclosed area with a permanently open section.

The first wall structure may be U shaped.

The removable structure may include a structure wall structure which partially blocks the open section of the semi enclosed area.

The second wall structure partially may block the permanently open section of the semi enclosed area such that there are two entrances into the semi enclosed area.

The system may further include an electronic scheduling system to schedule massages.

The electronic scheduling system may be mounted to the removable structure.

The electronic scheduling system may include at least one screen.

The electronic scheduling system may schedules massage appointments by discrete increments of time.

The electronic scheduling system may automatically schedule squeeze time between the massage appointments.

The massage appointments may be scheduled at the removable structure.

The massage appointments may be scheduled online.

The electronic scheduling system may pre-screen customers.

The electronic scheduling system may pre-screen by asking health related questions.

Messages may be communicated to a second screen mounted on the removable structure.

The invention also provides a system for massaging a person including a supporting apparatus for supporting the person in a relatively upright and face downward position for applying pressure to select locations on the person, wherein the supporting apparatus directly supports a face of the person; and an electronic scheduling system which schedules massages by discrete increments of time.

The electronic scheduling system may automatically schedules squeeze time between the massage appointments.

The massage appointments may be scheduled at the removable structure.

The massage appointments may be scheduled online.

The electronic scheduling system includes may include least one screen.

The invention also provides a system for massaging a person including a supporting a supporting apparatus for supporting the person in a relatively upright and face downward position for applying pressure to select locations on the person, wherein the supporting apparatus directly supports a face of the person; and an electronic scheduling system which pre-screens customers when scheduling massages.

The electronic scheduling system may pre-screen by asking health related questions.

The health related questions may inquire about fainting.

### BRIEF DESCRIPTION OF THE DRAWINGS

The invention is described by way of example with reference to the accompanying drawings, wherein:

FIG. 1 is a perspective view of a removable structure forming part of a system for massaging a person according to an embodiment of the invention.

FIG. 2 is an exterior view of a second wall structure forming part of the removeable structure including a advertising display panel and a touch screen.

FIG. 3 is an interior rear view of the second wall structure including a interior monitor and a cabinet.

FIG. 4 is a top view of the removable structure located within the removable structure.

FIG. 5 is a side view of a massage chair.

FIG. 6 is a top view of the massage chair.

FIG. 7 is a flow chart of an electronic scheduling system forming part of a system for massaging a person.

FIG. 8 is a welcome screen forming part of an electronic scheduling system.

FIG. 9 is a scheduling screen forming part of an electronic scheduling system.

FIG. 10 is a start time screen forming part of an electronic scheduling system.

FIG. 11 is a illustration of how the electronic scheduling system utilizes squeeze time.

FIG. 12 is an option screen forming part of an electronic scheduling system.

FIG. 13 is an information screen forming part of an electronic scheduling system.

FIG. 14 is a registration screen forming part of an electronic scheduling system.

FIG. 15 is a login screen forming part of an electronic scheduling system.

FIG. 16 is a warning message screen forming part of an electronic scheduling system.

FIG. 17 is a Zubio® massage questionnaire screen forming part of an electronic scheduling system.

FIG. 18 shows an acupuncture questionnaire screen forming part of an electronic scheduling system.

FIG. 19 shows a fainting screen forming part of an electronic scheduling system.

FIG. 20 shows a pain questionnaire screen forming part of an electronic scheduling system.

FIG. 21 shows a medication screen forming part of an electronic scheduling system.

FIG. 22 shows a payment screen forming part of an electronic scheduling system.

FIG. 23 shows a reservation confirmation screen forming part of an electronic scheduling system.

FIG. 24 is block diagram illustrating a computer system that can be used in the electronic scheduling system.

FIG. 25 is a block diagram illustrating a network that can be used in the electronic scheduling system.

### DETAILED DESCRIPTION OF THE INVENTION

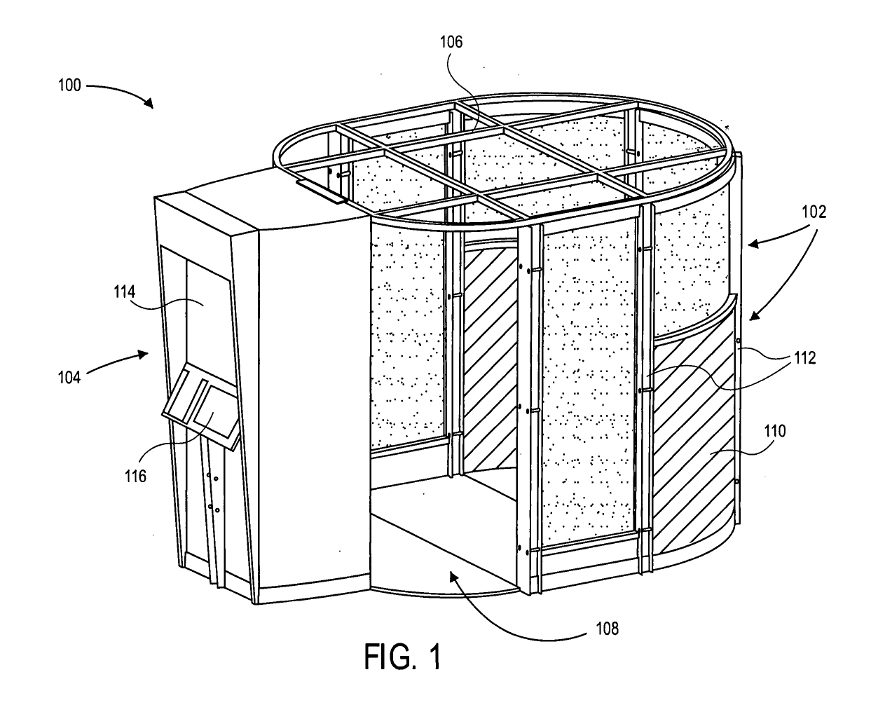

FIG. 1 of the accompanying drawings illustrates a removable structure 100 forming part of a system for massaging a person, according to an embodiment of the invention, including a first wall structure 102, and second wall structure 104, a roof structure 106, and a floor 108. The removable structure 100 may be housed within a larger permanent structure such as an airport or shopping mall, or alternatively placed temporarily outdoors. The removable structure 100 is removable in the sense that it may be disassembled so and removed without demolishing it, thus leaving it in intact and able to be reassembled at another location. The removable structure 100 is bolted to the floor 108 using large bolts. The removable structure 100 is removed by unbolting the large bolts. Alternatively the removable structure is an inflatable structure, constructed from flexible polymer wall and roof panels. The inflatable structure is attached to the floor using weights or bolts, or stakes in an outdoor setting. The inflatable structure is inflated with an air pump. The inflatable structure is deflated and stowed if it is removed or repositioned to a new location.

The removable structure 100 is a semi-private workspace with two entrances left permanently open. A customer receives a massage within the workspace from a trained massage specialist. The first wall structure 102 is in a semi-circle or “U” shaped pattern forming a partial enclosure with a partial permanent opening. The second wall structure 104 partially blocks the partial permanent opening thereby only allowing interior views from limited viewing angles.

The first wall structure 102 is constructed from panels 110 and frame 112. The panels 110 may be transparent, semi-transparent, or solid. Panels 110 may be glass, wood, metal, or various plastics. More or less transparent or semi-transparent panels may be used depending on the degree of privacy desired.

The roof structure 106 attaches to the top of the first wall structure 102 at the frame 112. The roof structure 106 also attaches to the second wall structure 104. The roof structure 106 is made of an open frame which allows light into the removable structure. The roof structure may also incorporate panels as described above. The roof structure may also incorporate lighting (not shown) and sound speakers (not shown).

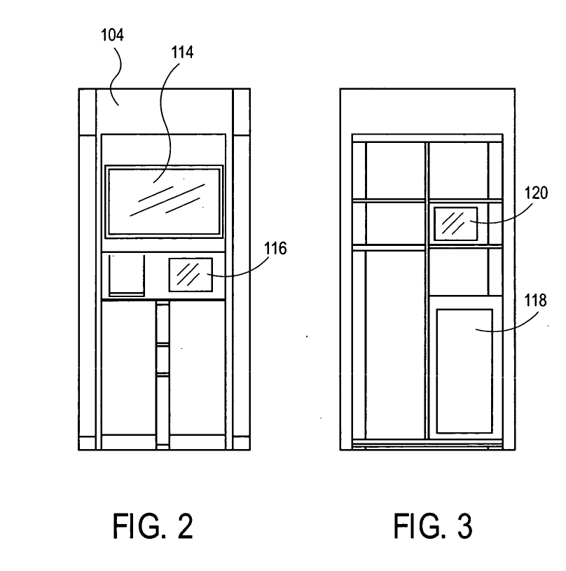

FIG. 2 shows the exterior view of the second wall structure 104. The second wall structure 104 includes an advertising display panel 114 and a touch screen 116. The advertising display panel 114 is a liquid crystal display (LCD) screen which displays a commercial message to persons walking past the removable structure. The touch screen 116 is part of an electronic scheduling system.

FIG. 3 shows the interior view of the second wall structure 104. The second wall structure includes a cabinet 118 and an interior monitor 120. Two computers are housed in the cabinet. The first computer is part of the electronic scheduling system and drives the touch screen 116. The first computer interfaced by massage customers. The second computer is part of the electronic scheduling system drives the interior monitor 120. The second computer is interfaced by the massage specialist.

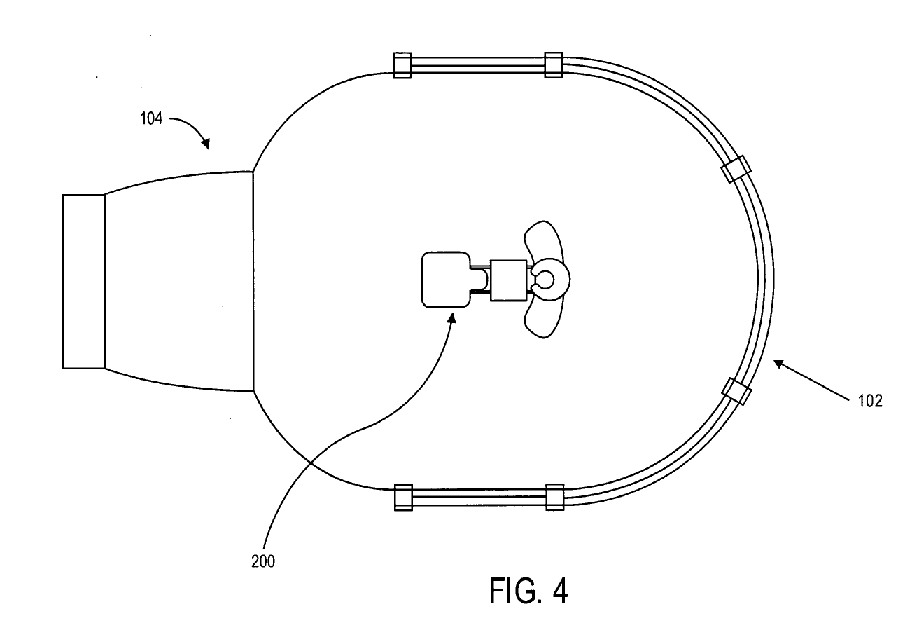

FIG. 4 shows a top view of the removable structure 100. The roof structure 106 is not shown. The removable structure also includes a massage chair 200. The massage chair 200 may be attached to the floor 108. Alternatively a portable massage chair may be used and stowed in the cabinet 118 when not in use. The massage chair is configured so that the customer will be facing away from the entrances and thus giving more privacy to the customer.

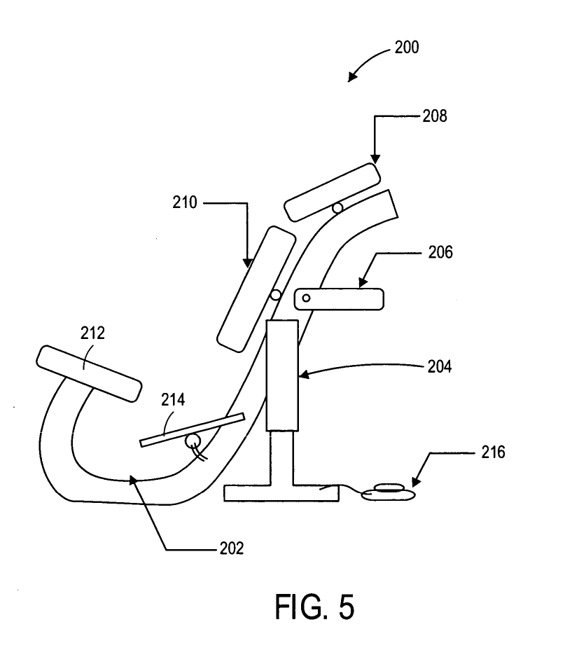

FIG. 5 shows a side view of the massage chair 200 forming part of a system for massaging a person. The massage chair supports the body of the customer in a relatively upright and face downward position. The face, hindquarters, front torso, forearms, and shins support the body weight, leaving the back portion of the customer exposed for massage. The massage chair includes a main frame 202. The main frame 202 is configured so that the bottom-most portion is open, and thus the customer can easily enter and exit the massage chair 200 without excessive lifting of legs. A height adjustable leg 204 is attached to the main frame 202. The height adjustable leg 204 may be mechanically, pneumatically or electrically adjustable. The height adjustable leg 204 is attached to the floor 108 and may also swivel in an axis perpendicular to the floor 108.

The customer sits in the massage chair 200 so that the customer is in a face-down and forward leaning position. The massage chair is adjustable to suit a variety of body sizes. The customer's forearms and elbows are supported by an adjustable padded arm rest 206. The customers face is supported by an adjustable open face rest 208, where the face sits inside an open halo of the face rest 208. The customer's front torso is supported by an adjustable chest rest 120. The customer's hindquarter region is supported by a seat pad 212. The customer's lower legs are supported by two adjustable leg supports 214. A foot pedal 216 allows the massage specialist to adjust the height of the massage chair 200.

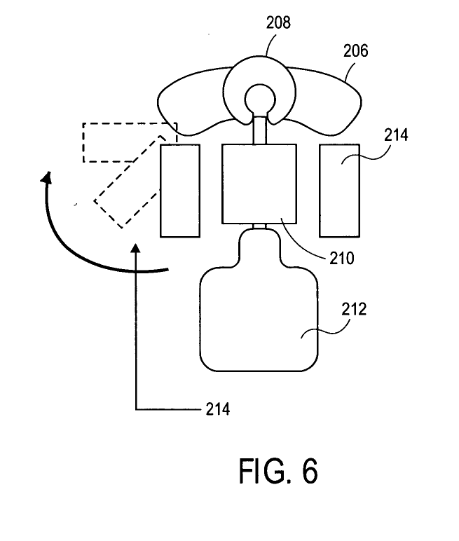

FIG. 6 shows a top view of the massage chair 200. The adjustable leg supports 214 swivels away from the massage chair to allow the customer easy entrance into and exit from the chair. The adjustable leg supports 214 are also removable.

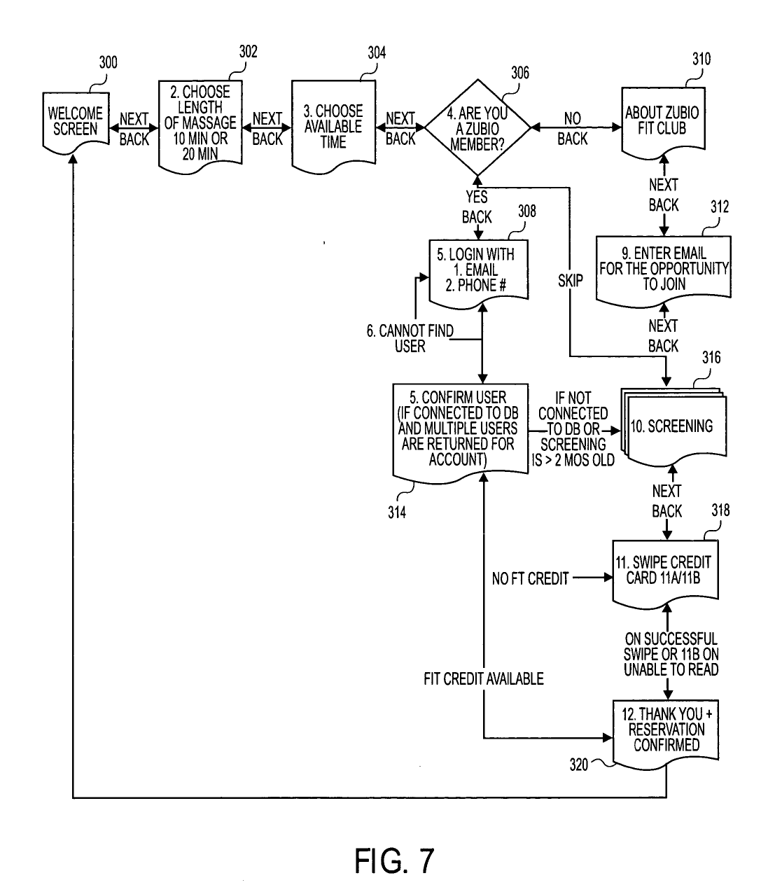

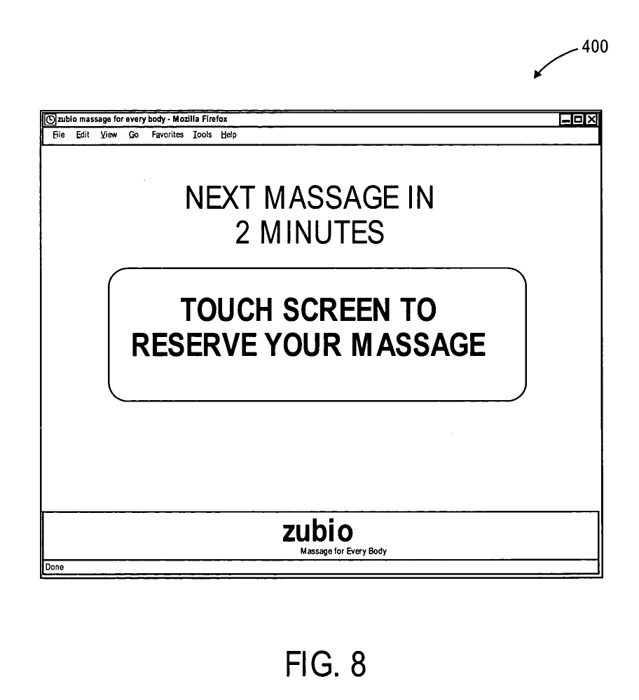

FIG. 7 shows a flow chart for an electronic scheduling system forming part of a system for massaging a person, which is implemented as a set of instructions on a computer system. In step 300 the touch screen 116 first displays a welcome screen message 400 as shown in FIG. 8. The welcome screen 400 displays when the next massage is available. The customer may touch a message on the screen to initiate the scheduling process. The electronic scheduling system may also incorporate music which may be chosen by the customer and played when the customer is receiving a massage.

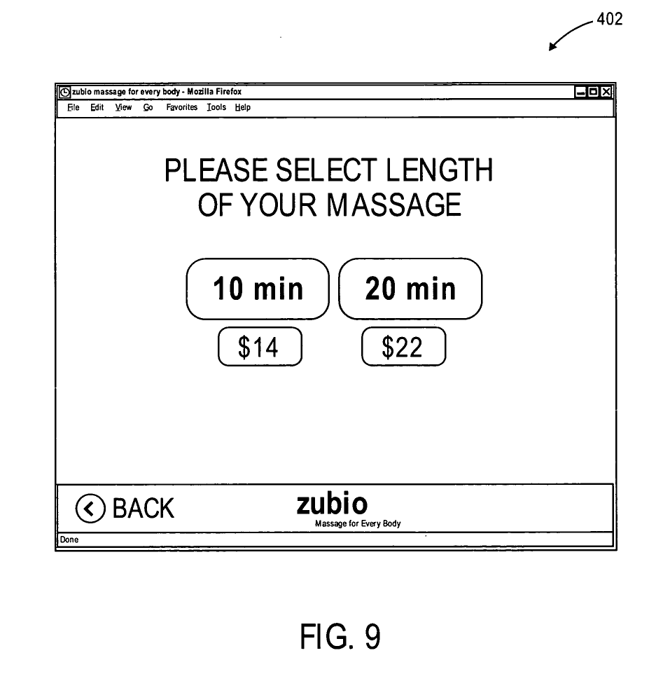

In step 302 a scheduling screen 402 as shown in FIG. 9 appears after the customer initiates the scheduling process. The scheduling screen 402 displays a massage length selection screen which allows the customer to choose the length of the massage in discrete time periods, for example 10, 20, or 30 minutes. Each massage length is displayed as a selectable button, with pricing displayed next to the button. The customer may optionally go back to the previous screen.

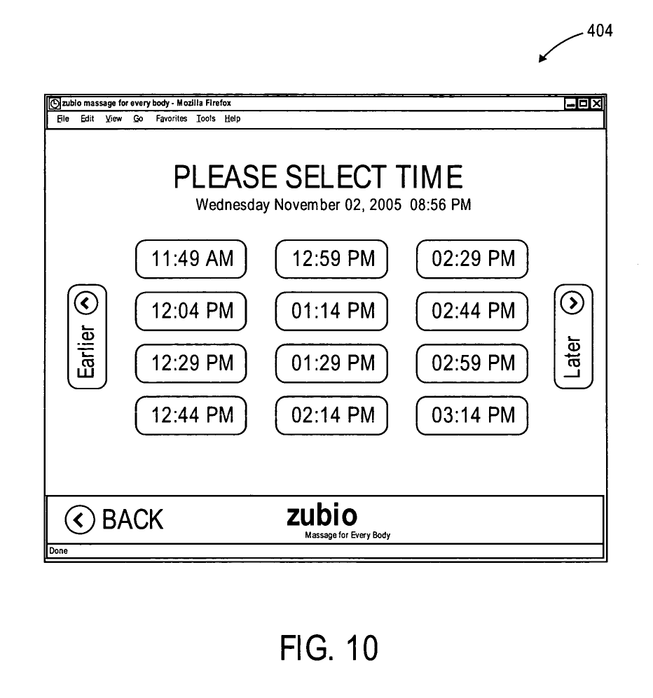

In step 304 a start time screen 404 as shown in FIG. 10 appears after the customer has selected a massage length. The start time screen 404 displays a plurality of start times, with each start time displayed as a selectable button. More or fewer start times will appear according depending on availability. The customer may optionally go back to the previous screen.

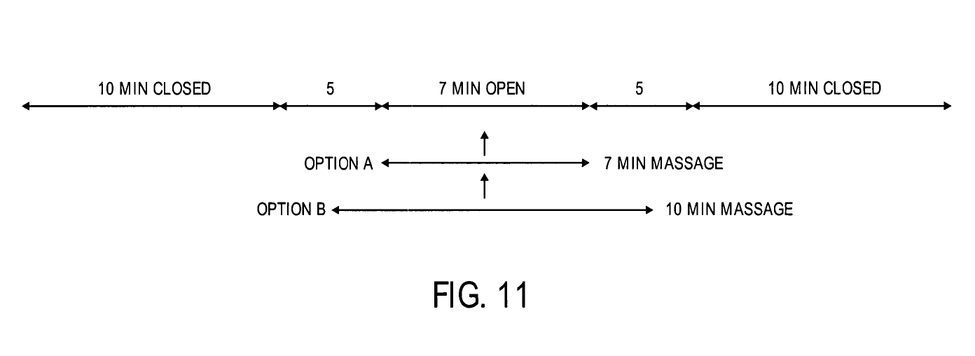

FIG. 11 shows how the electronic scheduling system schedules squeeze time. The electronic scheduling system automatically programs a buffer time between massages. Buffer time is an increment of time between massages which allows the massage specialist to clean the massage area and interact with the electronic scheduling system to view the needs of the next customer. Buffer time for example may be 5 minutes, thus if a 10 minute massage was scheduled at 12:00 PM, the electronic scheduling system would compute the next available appointment to be at 12:15 PM. FIG. 11 shows an open block of time corresponding to 7 minutes between two 10 minute scheduled massages. The electronic scheduling system allows a shortened massage of 7 minutes to be squeezed into the open block shown as option A. The electronic scheduling system will also reduce buffer time between massages in order to squeeze in a new massage as shown in option B.

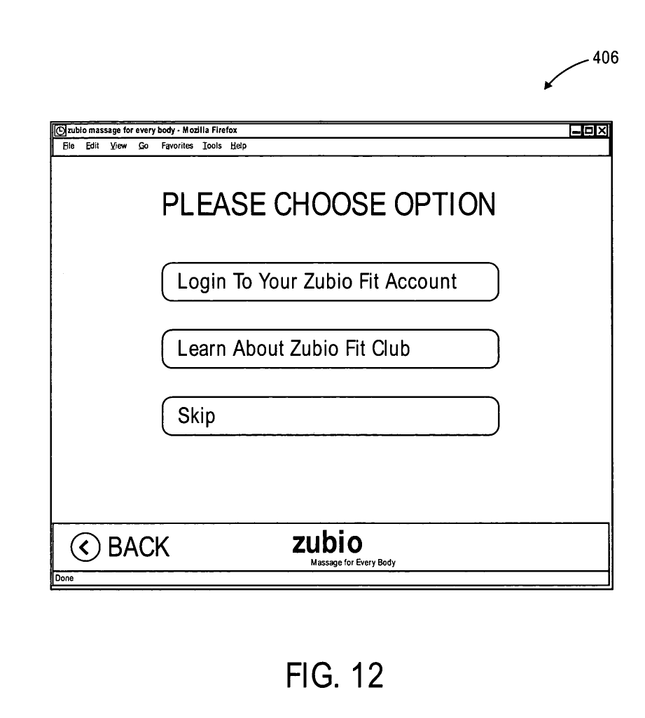

In step 306 an option screen 406 as shown in FIG. 12 appears after the customer has chosen a start time. The option screen 406 displays three options displayed as selectable buttons. The first option allows the customer to login to the customer's massage account. The second option allows the customer to learn more about creating a massage account. The third option allows the customer to skip the second option and go directly to screening as described later herein. The customer may optionally go back to the previous screen.

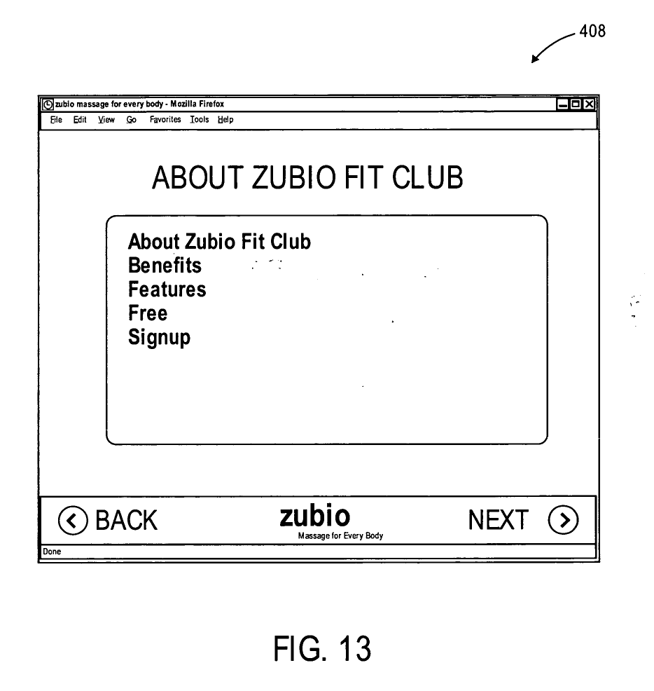

In step 310 an information screen 408 as shown in FIG. 13 appears if the user selects the second option on the option screen. The information screen 408 displays information about registering for a massage account, or joining the Zubio® Fit Club, including information about the benefits and features of the Zubio® Fit Club. The Zubio® Fit Club keeps a customer user profile which is an updatable personal file including billing information, credit-card information, music preferences, massage technique preferences, and massage chair 200 position preferences. Additional personal preferences not listed can be added to the personal file. If the customer does not wish to register or learn more, the customer may go back to the option screen 406 in step 306.

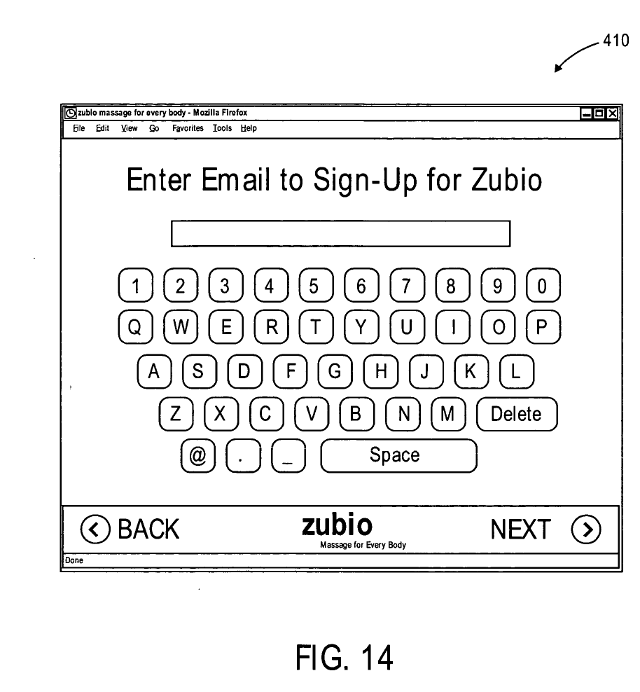

In step 312 a registration screen 410 as shown in FIG. 14 appears when the customer chooses to register for a massage account. The registration screen 410 allows the customer to enter personal information, including an email address. After completing registration and entering the email address a confirmation email is sent to validate the registration. If the customer does not wish to register, the customer may return to the information screen 408 in step 310.

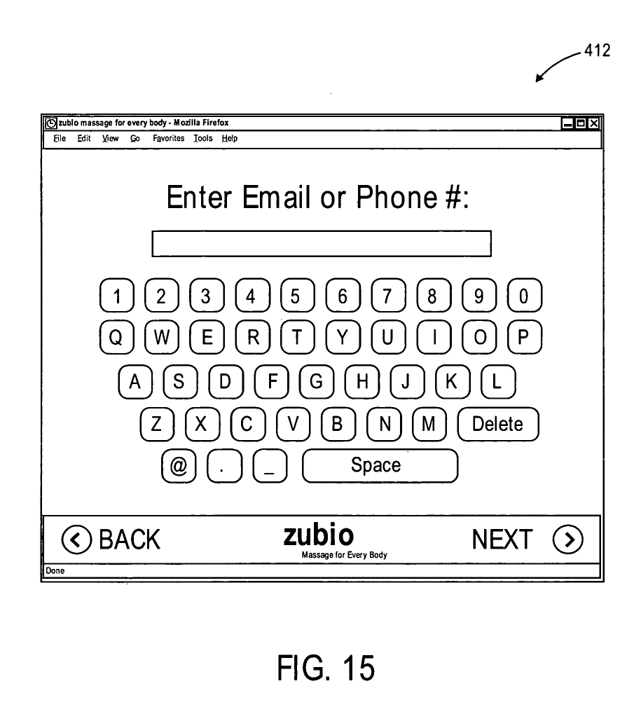

In step 308 a login screen 412 as shown in FIG. 15 appears when the customer selects the first option to login to the customer's massage account. The login screen 412 allows the customer to enter the customer's phone number or email address, after which the electronic scheduling system verifies the customer's account in step 314. Alternatively the customer logins using a personal identification number, a thumbscan on the touch screen, or using a magnetic or barcode scan card. A successful login will bypass pre-screening questions if the user has recently had a massage, for example two months. A successful login will bypass a payment step because the customer's account will automatically be billed.

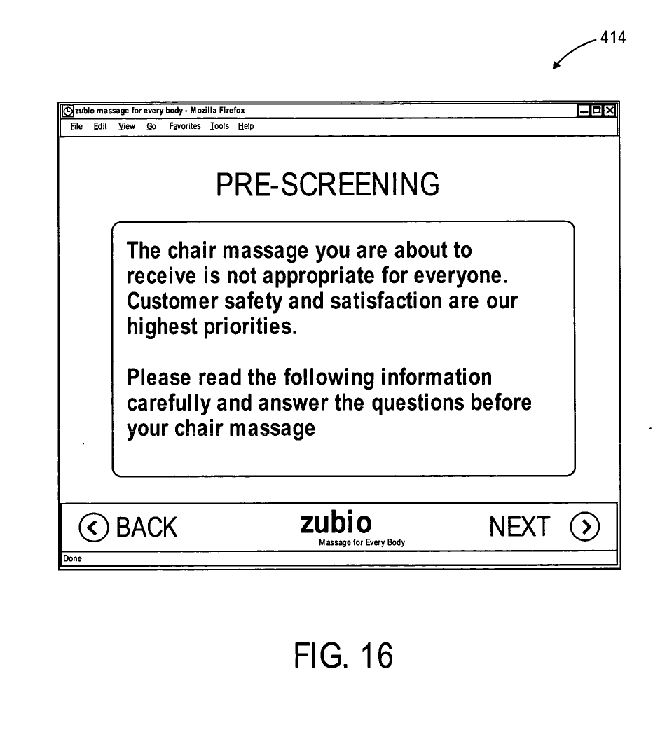

In step 316 a series of pre-screening screens as shown in FIGS. 16-21 appear after registering in step 312, or after the customer selects the third option in step 306. The pre-screening screen will also appear after a successful login in step 314 if the customer has not received a massage after an extended period of time, for example two months. The pre-screening screens ask a variety of questions in succession. These questions relate the general health and massage history of the customer. The questions can change over time and relate to a variety of subjects, and questions tailored to an individual customer can be asked, for example whether the customer is still experiencing a particular ailment noted in a prior massage.

FIG. 16 shows a warning message screen 414. The customer may go to the next pre-screening question after reading the warning message screen 414. The customer may optionally go back to the previous screen.

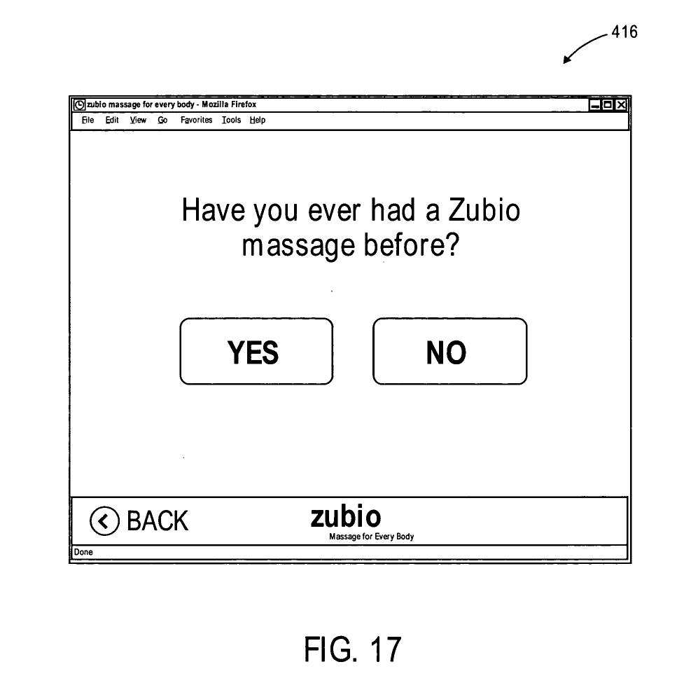

FIG. 17 shows a Zubio® massage questionnaire screen 416. The Zubio® massage questionnaire screen 416 inquires whether the customer has had a Zubio® massage before. The customer may optionally go back to the previous screen.

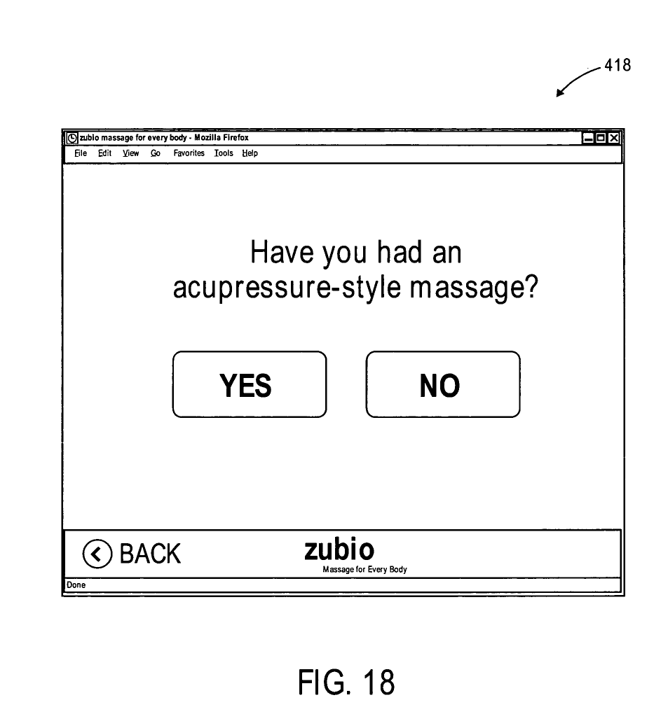

FIG. 18 shows an acupuncture questionnaire screen 418. The acupressure questionnaire screen 418 inquires whether the customer has had a acupressure massage before. The customer may optionally go back to the previous screen.

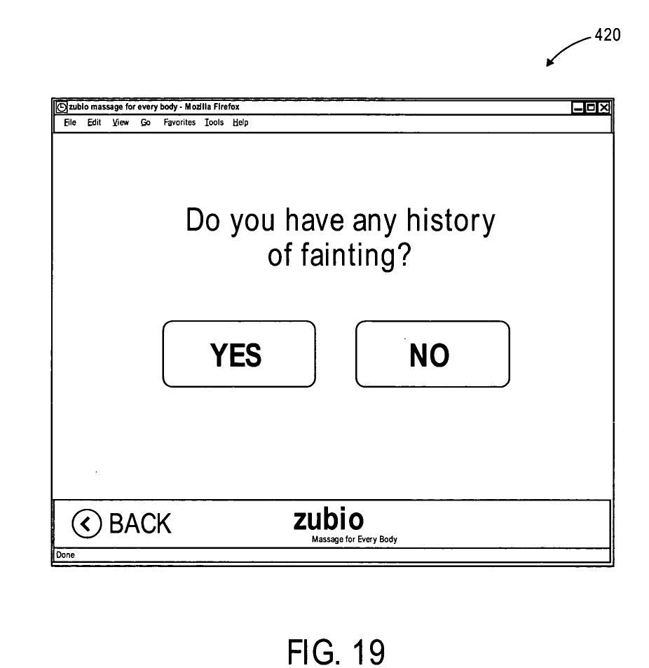

FIG. 19 shows a fainting screen 420. The fainting screen 420 inquires whether the customer has had a history of fainting. The customer may optionally go back to the previous screen.

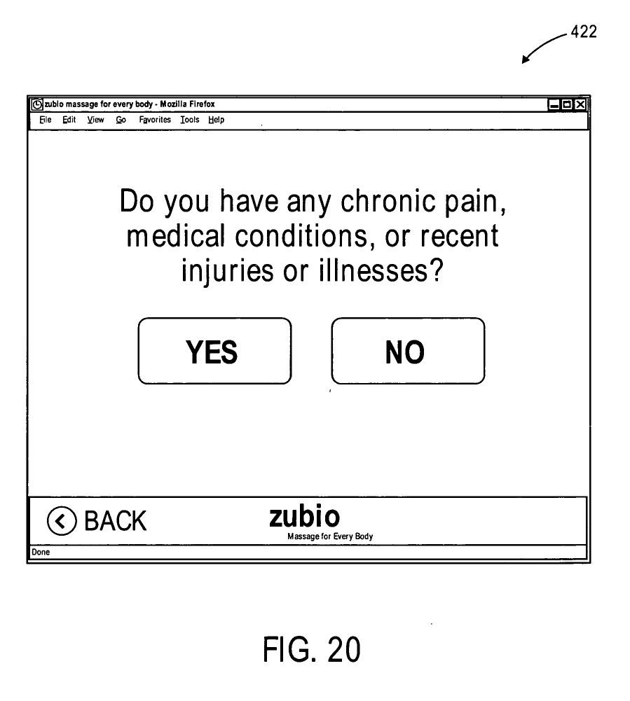

FIG. 20 shows a pain questionnaire screen 422. The pain questionnaire screen 422 inquires whether the customer has had any chronic pain, medical conditions, or recent injury or illnesses. The customer may optionally go back to the previous screen.

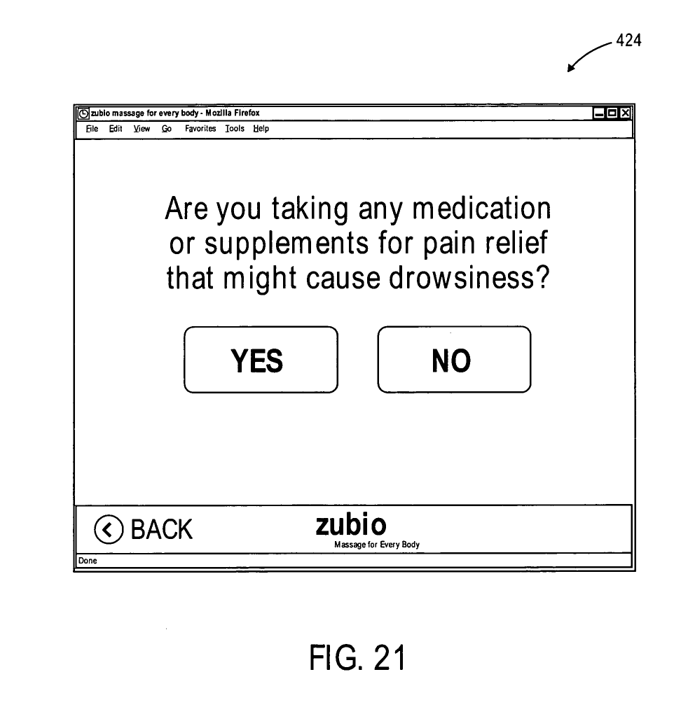

FIG. 21 shows a medication screen 424. The medication screen 424 inquires whether the customer is taking any medication or supplements for pain relief that might cause drowsiness. The customer may optionally go back to the previous screen. The results of the questions are communicated to the interior monitor 120.

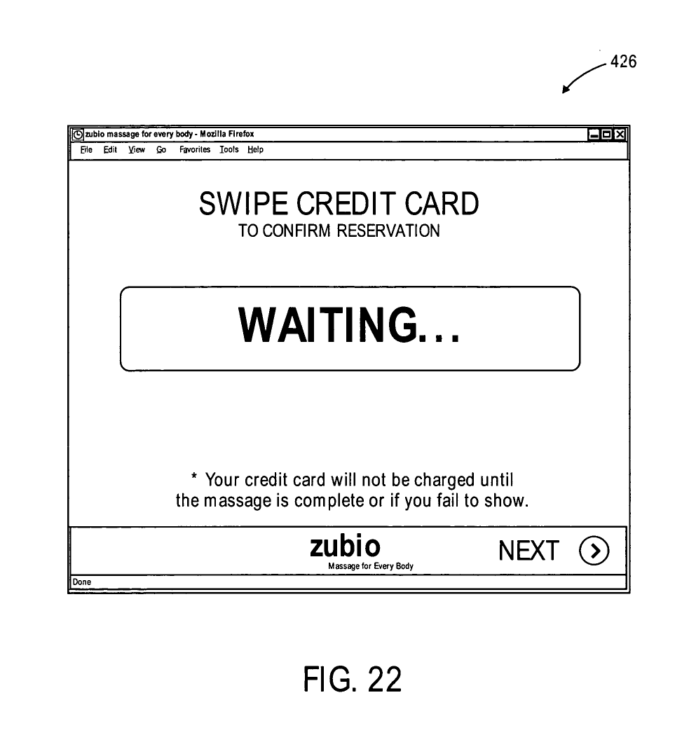

In step 318 a payment screen 426 as shown in FIG. 22 appears after the customer answers the series of pre-screening screens in step 316. The payment screen 426 will not appear if the customer answered the series of pre-screening screens after a successful login in step 314. The payment screen 426 instructs the customer to swipe a credit card on a credit card interface located about the touch screen 116. The electronic scheduling system will then read and bill the customer's credit card. If the swipe is not successful step 318 will be reinitiated.

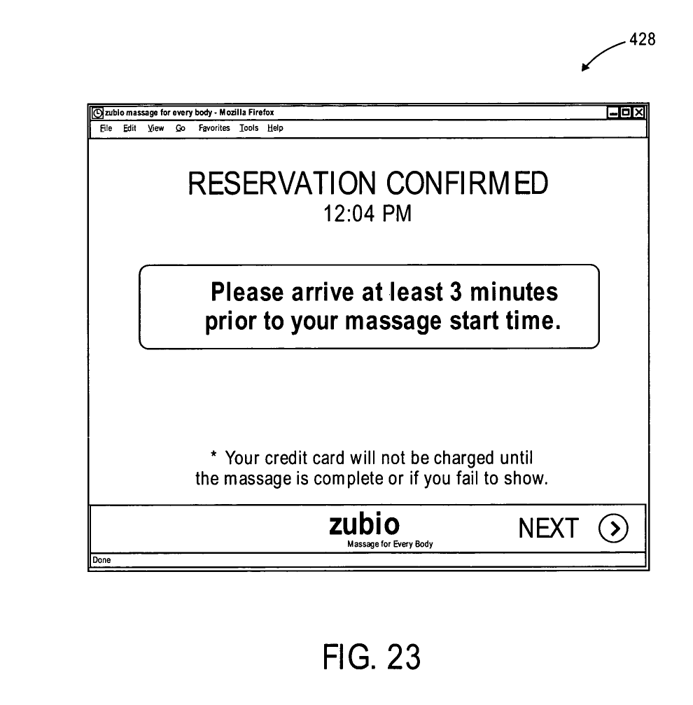

In step 320 a reservation confirmation screen 428 as shown in FIG. 23 appears after successful billing in step 318, or after a successful login in step 314. The reservation confirmation screen confirms the reservation to the customer. The welcome screen will reappear after a set amount of time, for example fifteen seconds, allowing a different customer to schedule a massage appointment. After a successful reservation is completed, reservation information is uploaded to a local server. The massage specialist views the reservation on the second computer which is connected to the local server. The massage specialist may cancel, alter, or schedule new massage appointments from the second computer. Alternatively the method described in FIG. 3A is performed remotely online, for example using a PDA, mobile phone, or home computer.

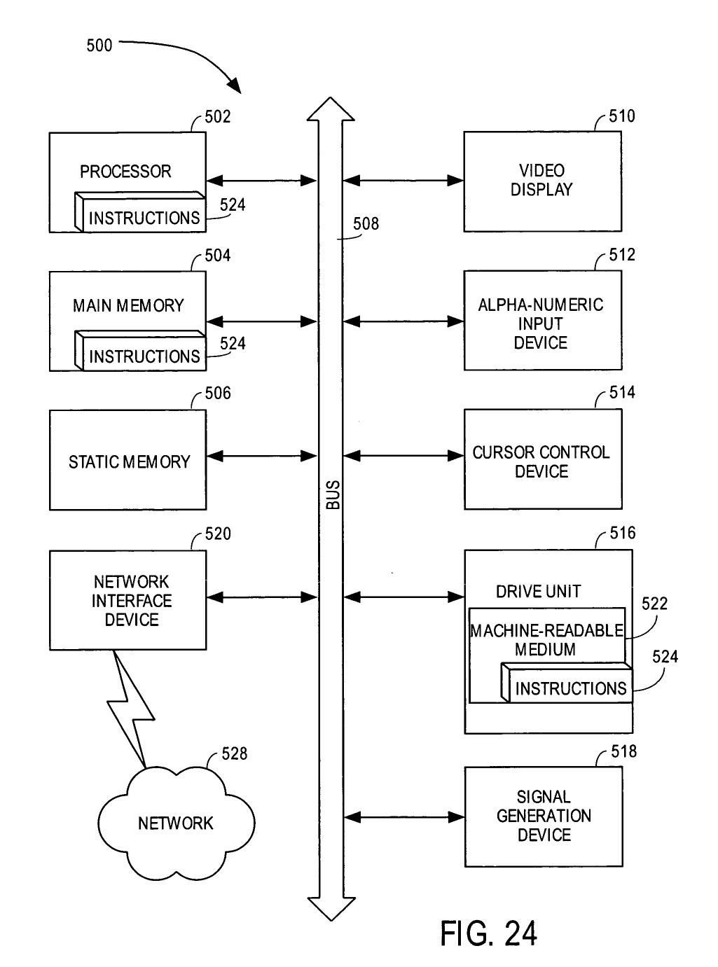

FIG. 24 shows a diagrammatic representation of a machine in the exemplary form of a computer system 500 within which a set of instructions, for causing the machine to perform any one or more methodologies may be executed. In alternative embodiments, the machine operates as a stand alone device or may be connected (e.g., network) to other networks. In a network deployment, the machine may operate in the capacity of a server or a client machine in a server-client network environment, or as peer machine in a peer-to-peer (or distributed) network environment. The machine may be a Personal Computer (PC), a tablet PC, a Set-Top Box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term (machine) shall also be taken to include any collection of machines that individually or jointly execute a set (or multiple sets) of instructions to perform any one or more of the methodologies discussed herein.

The exemplary computer system 500 includes a processor 502 (e.g., a Central Processing Unit (CPU), a Graphics Processing Unit (GPU) or both), a main memory 504 (e.g., Read Only Memory (ROM), flash memory, Dynamic Random Access Memory (DRAM) such as Synchronous DRAM (SDRAM) or Rambus DRAM (RDRAM), etc.), and a static memory 506 (e.g., flash memory, Static Random Access Memory (SRAM), etc.), which communicate with each other via a bus 508.

The computer system 500 may further include a video display 510 (e.g. Liquid Crystal Display (LCD) or a Cathode Ray Tube (CRT)). The computer system 500 also includes an alphanumeric input device 512 (e.g., a keyboard), a cursor control device 514 (e.g., a mouse), a disk drive unit 516, a signal generation device 518 (e.g. a speaker), and a network interface device 520.

The disk drive unit 516 includes a machine-readable medium 522 on which is stored one or more sets of instructions 524 (e.g. software) embodying any one or more methodologies or functions. The software may also reside, completely or at least partially, within the main memory 504 and/or within the processor 502 during execution thereof by the computer system 500, the main memory 504, and the processor 502 also constituting machine-readable media.

The software may further be transmitted or received over a network 528 via the network interface device 520.

While the machine-readable medium 524 is shown in an exemplary embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform one or more methodologies. The term “machine-readable medium” shall accordingly be taken to include, but not be limited to, solid-state memories, optical and magnetic media, and carrier wave signals.

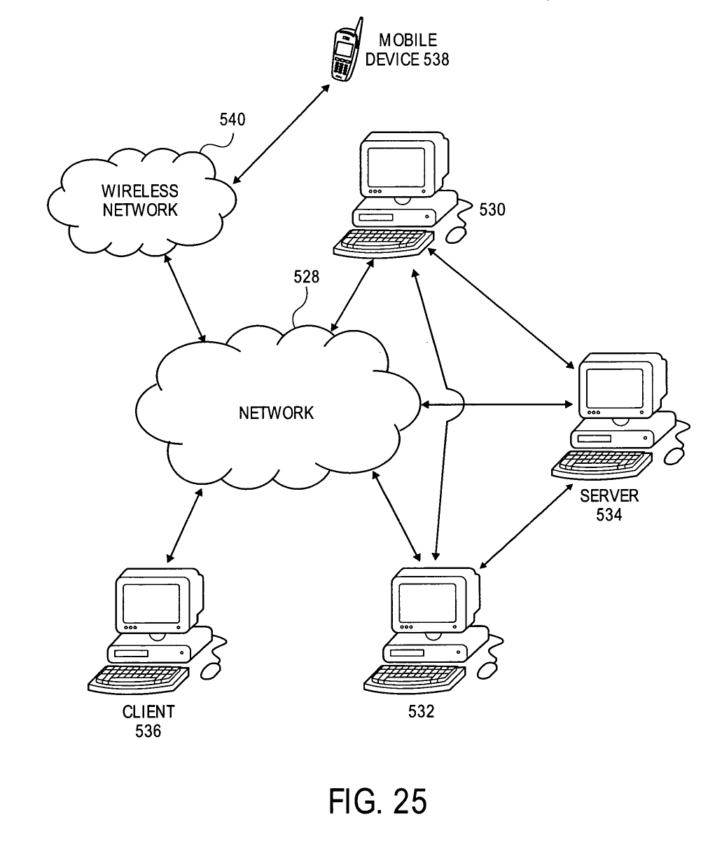

FIG. 25 shows various configurations of online connections. The first computer 530 and second computer 532 are connected to the network 528. The network 528 includes wide area networks (WAN), wireless networks, and local area networks (LAN). The first computer 530 and second computer 532 may be directly connected to the network 528 using system servers or connected through a local server computer 534. The first computer 530 and second computer 532 may be directly connected to each other in a peer to peer connection. Customers communicate with the second computer 530 through client computers 534 connected to the network. Customers may also communicate with the second computer 530 using a mobile device 538 such as a cellular phone, PDA, through a wireless network 540 connected to the network 528. The mobile device 538 and client computer 536 include a browser application to view the electronic scheduling system as a web page on the browser application.

While certain exemplary embodiments have been described and shown in the accompanying drawings, it is to be understood that such embodiments are merely illustrative and not restrictive of the current invention, that this invention is not restricted to the specific instructions and arrangements shown and described since modifications may occur to those ordinarily skilled in the art.

What is claimed:

1. A system for massaging a person comprising:
an intactly removable structure with at least a first wall structure defining at least a partial enclosure; and
a supporting apparatus within the enclosure for supporting the person in a relatively upright and face downward position for applying pressure to select locations on the person, wherein the supporting apparatus directly supports a face of the person.

2. The system of claim 1 wherein the first wall structure forms a semi enclosed area with an open section.

3. The system of claim 2 wherein the first wall structure is U shaped.

4. The system of claim 2 wherein the removable structure includes a second wall structure which partially blocks the open section of the semi enclosed area.

5. The system of claim 4 wherein the second wall structure partially blocks the permanently open section of the semi enclosed area such that there are two entrances into the semi enclosed area.

6. The system of claim 1 further comprising an electronic scheduling system to schedule massages.

7. The system of claim 6 wherein the electronic scheduling system is mounted to the removable structure.

8. The system of claim 7 wherein the electronic scheduling system includes at least one screen.

9. The system of claim 6 wherein the electronic scheduling system schedules massage appointments by discrete increments of time.

10. The system of claim 9 wherein the electronic scheduling system automatically schedules squeeze time between the massage appointments.

11. The system of claim 10 wherein massage appointments are scheduled at the removable structure.

12. The system of claim 10 wherein massage appointments are scheduled online.

13. The system of claim 9 wherein the electronic scheduling system pre-screens customers.

14. The system of claim 13 wherein the electronic scheduling system pre-screens by asking health related questions.

15. The system of claim 6 wherein messages are communicated to a second screen mounted on the removable structure.

16. A system for massaging a person comprising:
a supporting apparatus for supporting the person in a relatively upright and face downward position for applying pressure to select locations on the person, wherein the supporting apparatus directly supports a face of the person; and
an electronic scheduling system which schedules massages by discrete increments of time.

17. The system of claim 16 wherein the electronic scheduling system automatically schedules squeeze time between the massage appointments.

18. The system of claim 16 wherein massage appointments are scheduled at the removable structure.

19. The system of claim 16 wherein massage appointments are scheduled online.

20. The system of claim 7 wherein the electronic scheduling system includes at least one screen.

21. A system for massaging a person comprising:
a supporting apparatus for supporting the person in a relatively upright and face downward position for applying pressure to select locations on the person, wherein the supporting apparatus directly supports a face of the person; and
an electronic scheduling system which pre-screens customers when scheduling massages.

22. The system of claim 21 wherein the electronic scheduling system pre-screens by asking health related questions.

23. The system of claim 22 wherein at least one of the health related questions inquires about fainting.
